(19) United States
(12) Patent Application Publication
(10) Pub. No.: US 2009/0035326 A1
(43) Pub. Date: Feb. 5, 2009

(54) COMPOSITIONS WITH ANTIGENS ADSORBED TO CALCIUM PHOSPHATE

(75) Inventors: Mario Contorni, Siena (IT); Manmohan Singh, San Ramon, CA (US); Derek O'Hagan, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: Novartis AG, Basel (CH)

(21) Appl. No.: 12/092,379

(22) PCT Filed: Nov. 1, 2006
(86) PCT No.: PCT/US06/43162

§ 371 Date: Sep. 17, 2008

Related U.S. Application Data
(60) Provisional application No. 60/732,488, filed on Nov. 1, 2005.

(51) Int. Cl. A61K 39/385; A61K 47/04; A61P 31/04; A61P 31/16

(52) U.S. Cl. 424/196.11; 424/278.1; 424/197.11

(57) ABSTRACT

Calcium phosphate is used as an adjuvant, with a high degree of antigen adsorption to the adjuvant. The invention is particularly useful for adjuvanting conjugated capsular saccharide antigens. Buffers, such as phosphate or histidine buffers, can advantageously be used in combination with the calcium phosphate, and compositions may have a pH in the range of 5.5 to 7.5.

All documents cited herein are incorporated by reference in their entirety.

### TECHNICAL FIELD

This invention is in the field of vaccine adjuvants.

### BACKGROUND OF THE INVENTION

Aluminum salts are the most common adjuvants used in vaccines currently on the market. The adjuvanticity of these compounds was first discovered in 1926, and they are recognized as being safe by the FDA and international regulatory agencies. There is a move, however, to reduce the quantity of aluminum used in vaccines and to minimize the use of aluminum compounds as adjuvants.

It is an object of the invention to provide non-aluminum adjuvants for use in immunisation.

### SUMMARY OF THE INVENTION

The present invention relates to immunogenic compositions comprising: (i) an antigen; and (ii) a calcium phosphate salt, wherein at least 80% of the antigen is adsorbed to the calcium phosphate. In some embodiments, the calcium phosphate is amorphous. In other embodiments, the calcium phosphate is in particulate form. In a particular embodiment, the calcium phosphate has a calcium to phosphorus molar ratio between 1.35 and 1.83. In another particular embodiment, the concentration of calcium phosphate, measured as Ca⁺⁺, is between 0.1 mg/ml and 10 mg/ml. In certain embodiments, at least 90% of the antigen is adsorbed. In other embodiments, at least 95% of the antigen is adsorbed. In still other embodiments, at least 99% of the antigen is adsorbed.

The immunogenic compositions of the invention can include one or more further adjuvants and/or immunostimulatory agents. In a particular embodiment, the immunogenic compositions of the invention include an immunostimulatory oligonucleotide. In another particular embodiment, the immunogenic compositions of the invention are substantially free from aluminium salts.

The antigen can be a bacterial or viral antigen. In certain embodiments, the antigen is a conjugated bacterial capsular saccharide. The capsular saccharide can be from H. influenzae type B, N. meningitidis, S. pneumoniae, for example. In certain other embodiments, the antigen is an influenza virus antigen. The influenza virus can be a pandemic strain.

In a particular embodiment, the immunogenic compositions of the invention include NaCl. In other embodiments, the immunogenic compositions of the invention have an osmolality between 200 mOsm/kg and 400 mOsm/kg.

In another embodiment, the immunogenic compositions of the invention include a buffer. In a particular embodiment, the immunogenic compositions of the invention include a histidine buffer.

In other embodiments, the immunogenic compositions of the invention have a pH between 5.5 and 7.5. In further embodiments, the immunogenic compositions of the invention are free from mercury.

The present invention also relates to adjuvant compositions comprising: (i) a calcium phosphate salt; and (ii) an adjuvant selected from the group consisting of: 3D-MPL, immunostimulatory oligonucleotides, and imidazoquinolones; wherein at least 50% of the adjuvant is adsorbed to the calcium phosphate.

### DETAILED DESCRIPTION OF THE INVENTION

The invention is based on the use of calcium phosphate as an adjuvant, with a high degree of antigen adsorption to the adjuvant. The invention is particularly useful for adjuvanting conjugated capsular saccharide antigens. Buffers, such as phosphate or histidine buffers, can advantageously be used in combination with the calcium phosphate, and compositions may have a pH in the range of 5.5 to 7.5.

Thus the invention provides an immunogenic composition comprising: (i) an antigen; and (ii) a calcium phosphate salt, wherein at least 80% (by weight) of the antigen is adsorbed to the calcium phosphate.

The invention also provides a method for preparing an immunogenic composition comprising the step of mixing an antigen and a calcium phosphate salt, whereby at least 80% (by weight) of the antigen becomes adsorbed to the calcium phosphate.

### The Calcium Phosphate Salt

The use of calcium phosphate as a vaccine adjuvant was disclosed as long ago as 1957 [1], with further studies being published in 1969 [2]. Several further studies have been published since then, and hydrated calcium phosphate gel adjuvant has been available from Superfos (Vedbaek, Denmark) for several years.

Chapter 8 of reference 3 reviewed calcium phosphate adjuvants in 1995. Antigens can be adsorbed to calcium phosphate either by in situ precipitation of the salt in the presence of the antigens or by adsorption to a pre-formed salt. Commercial sources of pre-formed calcium phosphate gel are mentioned. Details are given on the effect of precipitation conditions on physicochemical characteristics of the adjuvant, including adsorption capacity.

Reference 4 reports on the structure and adsorption properties of various calcium phosphate adjuvants. Rather than being strict Ca₃(PO₄)₂, the adjuvants were reported to be non-stoichiometric hydroxyapatite of formula Ca₁₀₋ₓ(HPO₄)ₓ(PO₄)₆₋ₓ(OH)₂₋ₓ and a pH-dependent surface charge with a point of zero charge (PZC) of 5.5. The adjuvants can form needle-like particles having dimensions of approximately 10 nm×150 nm as well as irregularly shaped plates having diameters of approximately 20-30 nm.

Reference 5 discloses a reactive amorphous calcium phosphate, containing reactive vacant sites, the reactive sites having been obtained by removal of a carbonate pre-component of carbonated amorphous calcium phosphate by thermal decomposition of the pre-component into gaseous or vaporous by-products.

References 6 & 7 disclose a particulate calcium phosphate adjuvant, wherein the particle has a diameter in the range of 300-4000 nm (nanoparticle) and has a spherical shape and a smooth surface. Reference 8 discloses that these particles can be used for mucosal immunization.

Mucosal immunization is also disclosed in reference 9, where a method for vaccinating a mammal to cause an IgA antibody response uses particulate hydroxylated calcium phosphate of a size suitable for transport across epithelium.

Reference 10 discloses composite particles that are soluble in vivo and which comprise a particle of a polymeric substance having a calcium phosphate compound having a Ca/P ratio of about 1.0 to 2.0 coated on its surface.

Reference 11 discloses an injectable aqueous gel of calcium phosphate for adsorbing vaccines, wherein calcium and phosphate ions are combined in proportions such that the weight ratio Ca/P is from 1.62 to 1.85, and such that the settling time of the gel when containing 0.07 atom Ca per liter is between 1-20 mm in 10 minutes at 20° C.

The Ca to P molar ratio of calcium phosphate adjuvants can vary e.g. between 1.35 and 1.83 [see chapter 8 of ref 3]. The adsorption properties of the adjuvant have been found to vary depending on the conditions used during precipitation e.g. slow mixing gave an adjuvant with lower adsorption capacity that an adjuvant formed by quick mixing.

All of these various forms of calcium phosphate can be used with the invention.

The amount of calcium phosphate, measured as Ca⁺⁺, may be between 0.1 mg/ml and 10 mg/ml e.g. between 0.5-5 mg/ml, preferably 0.75-3 mg/ml, 0.9-1.5 mg/ml, or about 1 mg/ml.

The calcium phosphate adjuvant has the capacity to adsorb antigens. For a given antigen, at least 80% (e.g. ≧85%, ≧90%, ≧92.5%, ≧95%, ≧97.5%, ≧97.5%, ≧98%, ≧99%, ≧99.5%, etc.) by weight of the total amount of that antigen is adsorbed. As calcium phosphate adjuvants are insoluble, typically particulate, the degree of adsorption can conveniently be measured by a method involving centrifugation and then determination of the amount of antigen in one (or both) of the solid or soluble material. Unadsorbed antigen will remain in solution after centrifugation. For example, the adsorption capacity of calcium phosphate adjuvants was measured by this method in reference 12. Adsorption of diphtheria and tetanus toxoids to 1 mg of Ca⁺⁺ was incomplete when (a) diphtheria toxoid levels rose above 100 Lf and (b) tetanus toxoid levels rose above 25 Lf.

For adsorption, a calcium phosphate adjuvant is preferably used in the form of an aqueous suspension to which the antigen HBsAg is added. The calcium salt can be diluted to the required concentration before addition of the antigen.

### Further Adjuvants

As well as including a calcium phosphate adjuvant, compositions of the invention may include one or more further adjuvants and/or immunostimulatory agents. For example, reference 13 discloses the use of an amorphous calcium phosphate adjuvant that can be mixed with further adjuvants, and reference 14 discloses an adjuvant formulation having calcium phosphate in the aqueous phase of an oil-in-water emulsion.

Further components for inclusion in the compositions include, but are not limited to:
Immunostimulatory oligonucleotides. These include ‘CpG oligonucleotides’ i.e. nucleic acids that include nucleotide sequences containing a CpG motif (a dinucleotide sequence containing an unmethylated cytosine followed by guanosine). Bacterial double stranded RNA or oligonucleotides containing palindromic or poly(dG) sequences have also been shown to be immunostimulatory. The CpG's can include nucleotide modifications/analogs such as phosphorothioate modifications and can be double-stranded or single-stranded. Optionally, the guanosine may be replaced with an analog such as 2′-deoxy-7-deazaguanosine. Refs 15-17 give examples of possible analog substitutions. The adjuvant effect of CpG oligonucleotides is further discussed in references 18-23.

The CpG sequence may be directed to TLR9, such as the motif GTCGTT or TTCGTT [24]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 25-27. Preferably, the CpG is a CpG-A ODN.

Preferably, the CpG oligonucleotide is constructed so that the 5′ end is accessible for receptor Preferably, the CpG oligonucleotide is constructed so that the 5′ end is accessible for receptor recognition. Optionally, two CpG oligonucleotide sequences may be attached at their 3′ ends to form “immunomers”. See, for example, refs. 24 & 28-30.
Aminoalkyl glucosaminide phosphate (AGP) derivatives, including RC-529 [31-34], which is an aminoalkyl glucosaminide 4-phosphate sold by Corixa Corporation.
A monophosphoryl lipid A (MPL) analog, such as 3-de-O-acylated MP (‘3D-MPL’). The 3D-MPL adjuvant can also be adsorbed onto the calcium phosphate. 3D-MPL can be prepared from a heptoseless mutant of Salmonella minnesota, and is chemically similar to lipid A but lacks an acid-labile phosphoryl group and a base-labile acyl group. Preparation of 3D-MPL was originally described in reference 35, and the product has been manufactured and sold by Corixa Corporation under the trade name MPL™. Further details can be found in references 36 to 39. 3D-MPL can take the form of a mixture of related molecules, varying by their acylation (e.g. having 3, 4, 5 or 6 acyl chains, which may be of different lengths). Other suitable lipid A derivatives include derivatives of lipid A from Escherichia coli such as OM-174 [refs. 40 & 41].
Oil-in-water emulsions, such as squalene-in-water emulsions. For example, the MF59 adjuvant [Chapter 10 of ref. 3; see also ref. 42] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.
A saponin. Saponins are a heterologous group of sterol glyco sides and triterpenoid glyco sides that are found in the bark, leaves, stems, roots and even flowers of a wide range of plant species. Saponin from the bark of the Quillaia saponaria Molina tree have been widely studied as adjuvants. Saponin can also be obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 43. Saponin formulations may also comprise a sterol, such as cholesterol [44].

Combinations of saponins and cholesterols can be used to form unique particles called immunostimulating complexs (ISCOMs) [chapter 23 of ref. 3]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 44-46. Optionally, the ISCOMS may be devoid of additional detergent [47].

A review of the development of saponin based adjuvants can be found in refs. 48 & 49.
Virosomes and virus-like particles (VLPs). These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 50-55. Virosomes are discussed further in, for example, ref. 56.
ADP-ribosylating toxins and detoxified derivatives thereof. The protein may be derived from E. coli (E. coli heat labile enterotoxin “LT”), cholera (“CT”), or pertussis (“PT”). The use of detoxified ADP-ribosylating toxins as mucosal adjuvants is described in ref. 57 and as parenteral adjuvants in ref. 58. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 59-66. Numerical reference for amino acid substitutions is preferably based on the alignments of the A and B subunits of ADP-ribosylating toxins set forth in ref. 67, specifically incorporated herein by reference in its entirety.
Human immunomodulators. Human immunomodulators suitable for use as adjuvants in the invention include cytokines, such as interleukins (e.g. IL-1, IL-2, IL-4, IL-5, IL-6, IL-7, IL-12 [68], etc.) [69], interferons (e.g. interferon-γ), macrophage colony stimulating factor, and tumor necrosis factor.
Bioadhesives and Mucoadhesives. Suitable bioadhesives include esterified hyaluronic acid microspheres [70] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [71].
Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).
Liposomes (Chapters 13 & 14 of ref. 3). Examples of liposome formulations suitable for use as adjuvants are described in refs. 72-74.
Polyoxyethylene ether and polyoxyethylene ester formulations [75]. Such formulations further include polyoxyethylene sorbitan ester surfactants in combination with an octoxynol [76] as well as polyoxyethylene alkyl ethers or ester surfactants in combination with at least one additional non-ionic surfactant such as an octoxynol [77]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.
Polyphosphazene (PCPP) e.g. as described in refs. 78 and 79.
Muramyl peptides, such as N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).
Imidazoquinolone compounds. Examples of imidazoquinolone compounds suitable for use adjuvants in the invention include Imiquamod (“R-837”) [80,81] and its homologues (e.g. “Resiquimod 3M”, “R-848” [82]), including salts thereof (e.g. the hydrochloride salts). Further details about immunostimulatory imidazoquinolines can be found in references 83 to 87.

Adsorption of these further adjuvants to the calcium phosphate is useful. Adsorption of 3D-MPL, immunostimulatory oligonucleotides and imidazoquinolones can facilitate their localised presentation to the immune system. Adjuvants and antigens may both be adsorbed to the calcium phosphate, which may be achieved by simultaneous or sequential adsorption steps. As an alternative, adjuvants and antigens may be separately adsorbed to different batches of salt, and may then be mixed. Moreover, these adsorbed adjuvants are useful in their own right, and so the invention provides a composition comprising: (i) a calcium phosphate salt; and (ii) one or more of the above-mentioned further adjuvants, wherein at least 50% (e.g. ≧60%, ≧70%, ≧80%, ≧90%, ≧95% or substantially 100%) of the further adjuvant is adsorbed to the calcium phosphate. This composition may also include an antigen (as described elsewhere herein). The composition may also include a liquid carrier e.g. an oil-in-water emulsion.

Compositions of the invention are preferably substantially free from aluminium salts.

### The Antigen

Immunogenic compositions of the invention include one or more antigens. Where a single antigen is present, at least 80% is adsorbed to the calcium phosphate. Where more than one antigen is present, at least 80% of one of the antigens is adsorbed to the calcium phosphate, and the other antigen(s) may or may not be adsorbed to the calcium phosphate. Preferably, however, at least 80% of each of the antigens is adsorbed.

The antigen(s) may be derived from bacteria, viruses or fungi. Typical antigens for inclusion in the compositions of the invention include, but are not limited to:
Diphtheria toxoid (‘Dt’), disclosed in more detail in chapter 13 of reference 88. Preferred diphtheria toxoids are those prepared by formaldehyde treatment. Quantities of diphtheria toxoid can be expressed in international units (IU). For example, the NIBSC supplies the ‘Diphtheria Toxoid Adsorbed Third International Standard 1999’ [89,90], which contains 160 IU per ampoule. As an alternative to the IU system, the ‘Lf’ unit (“flocculating units” or the “limes flocculating dose”) is defined as the amount of toxoid which, when mixed with one International Unit of antitoxin, produces an optimally flocculating mixture [91]. For example, the NIBSC supplies ‘Diphtheria Toxoid, Plain’ [92], which contains 300 LF per ampoule, and also supplies ‘The 1st International Reference Reagent For Diphtheria Toxoid For Flocculation Test’ [93] which contains 900 Lf per ampoule. The concentration of diphtheria toxoid in a composition of invention is typically at least 50 IU/ml.
Tetanus toxoid (‘Tt’), disclosed in more detail in chapter 27 of reference 88. Preferred tetanus toxoids are those prepared by formaldehyde treatment. Quantities of tetanus toxoid can be expressed in international units (IU). For example, the NIBSC supplies the ‘Tetanus Toxoid Adsorbed Third International Standard 2000’ [94,95], which contains 469 IU per ampoule. As an alternative to the IU system, the ‘Lf’ unit (“flocculating units” or the “limes flocculating dose”) is defined as the amount of toxoid which, when mixed with one International Unit of antitoxin, produces an optimally flocculating mixture [91]. For example, the NIBSC supplies ‘The 1st International Reference Reagent for Tetanus Toxoid For Flocculation Test’ [96] which contains 1000 Lf per ampoule. The concentration of tetanus toxoid in a composition of the invention is typically at least 100 IU/ml.
Cellular Bordetella pertussis antigen, typically in the form of inactivated B. pertussis cells. Preparation of cellular pertussis antigens is well documented [e.g. see chapter 21 of ref. 88]. Quantities of wP antigens can be expressed in international units (IU). For example, the NIBSC supplies the ‘Third International Standard For Pertussis Vaccine’ [97], which contains 46 IU per ampoule. Each ampoule contains the freeze-dried residue of 2.0 ml aliquots of an aqueous solution which contained 10 liters of bacterial suspension (equivalent to 180 opacity units in terms of the U.S. Opacity Standard) diluted with eight litres of M/15 Sorensen's buffer pH 7.0. As an alternative to the IU system, the ‘OU’ unit (“opacity units”) is also used (e.g. 4 OU may be about 1 IU). There will typically be at least 8 IU/ml.
Acellular Bordetella pertussis antigen, including one or more of pertussis toxoid (PT), filamentous haemagglutinin (FHA), pertactin (also known as the ‘69 kiloDalton outer membrane protein’), and fimbriae (e.g. agglutinogens 2 and 3). The invention preferably uses at least two of, and preferably all three of, PT, FHA and pertactin (i.e. without using fimbriae). FHA and pertactin may be treated with formaldehyde prior to use according to the invention. PT is preferably detoxified by treatment with formaldehyde and/or glutaraldehyde. As an alternative to this chemical detoxification procedure the PT may be a mutant PT in which enzymatic activity has been reduced by mutagenesis [98], but detoxification by chemical treatment is preferred. Quantities of acellular pertussis antigens are typically expressed in micrograms. There will typically be between 25-75 μg PT, about 25-75 μg FHA and about 10-20 μg pertactin per dose.
Hepatitis B virus surface antigen (‘HBsAg’). A typical HBsAg will be expressed by recombinant DNA methods in a yeast, such as a Saccharomyces cerevisiae, Pichia pastoris or Hanensula polymorpha. The HBsAg is preferably non-glycosylated. It may take the form of substantially-spherical particles including a lipid matrix comprising phospholipids and, optionally, phosphatidylinositol. The HBsAg is preferably from HBV subtype adw2. There will typically be between 1 and 50 μg HBsAg.
Hepatitis A virus antigen (‘HAV’), as disclosed in chapter 15 of reference 88. A preferred HAV component is based on inactivated virus, and inactivation can be achieved by formalin treatment. Virus can be grown on human embryonic lung diploid fibroblasts, such as MRC-5 cells. A preferred HAV strain is HM175, although CR326F can also be used. The cells can be grown under conditions that permit viral growth. The cells are lysed, and the resulting suspension can be purified by ultrafiltration and gel permeation chromatography. The amount of HAV antigen, measured in EU (Elisa Units), is typically at least 600 EU/ml.
An inactivated poliovirus antigen (‘IPV’), as disclosed in chapter 24 of reference 88. Polioviruses may be grown in cell culture, and a preferred culture uses a Vero cell line, derived from monkey kidney. Vero cells can conveniently be cultured microcarriers. After growth, virions may be purified using techniques such as ultrafiltration, diafiltration, and chromatography. Prior to administration to patients, polioviruses must be inactivated, and this can be achieved by treatment with formaldehyde. Poliomyelitis can be caused by one of three types of poliovirus. The three types are similar and cause identical symptoms, but they are antigenically very different and infection by one type does not protect against infection by others. It is therefore preferred to use three poliovirus antigens: poliovirus Type 1 (e.g. Mahoney strain), poliovirus Type 2 (e.g. MEF-1 strain), and poliovirus Type 3 (e.g. Saukett strain). The viruses are preferably grown, purified and inactivated individually, and are then combined to give a bulk trivalent mixture for use with the invention. Quantities of IPV are typically expressed in the ‘DU’ unit (the “D-antigen unit” [99]). The amount of IPV antigen depends on the strain serotype. For a type 1 virus, a composition typically contains about 80 DU/ml. For a type 2 virus, a composition typically contains about 16 DU/ml. For a type 3 virus, a composition typically contains about 65 DU/ml.
An influenza virus antigen, as described in more detail in chapters 17 & 18 of reference 88. Broadly, influenza virus vaccines can be based on live virus or inactivated virus, and inactivated vaccines can be based on whole virus, split virus or on purified surface antigens (including hemagglutinin ‘HA’ and neuraminidase ‘NA’ glycoproteins). The viruses used to prepare the vaccines can be grown either on eggs or, preferably, on cell culture [100]. When grown on cell culture, the composition preferably contains less than long (preferably less than 1 ng, and more preferably less than 100 pg) of residual host cell DNA per dose, although trace amounts of host cell DNA may be present. Contaminating DNA can be removed during vaccine preparation using standard purification procedures e.g. chromatography, etc. Removal of residual host cell DNA can be enhanced by nuclease treatment e.g. by using the Benzonase™ DNase [101]. Suitable cell lines for influenza virus production include Vero cells, MDCK cells and PER.C6 cells. When an influenza vaccine has been prepared using a cell culture method, the composition is preferably free from: reoviruses (particularly mammalian); polyomaviruses; bimaviruses; circoviruses; parvoviruses; and herpes simplex viruses.

Vaccine strains for influenza virus change from season to season. In the current inter-pandemic period, vaccines typically include two influenza A strains (H1N1 and H3N2) and one influenza B strain, and trivalent vaccines are typical. The invention may also use viruses from pandemic strains (i.e. strains to which the vaccine recipient and the general human population are immunologically naïve), such as H2, H5, H7 or H9 subtype strains (in particular of influenza A virus), and influenza vaccines for pandemic strains may be monovalent or may be based on a normal trivalent vaccine supplemented by a pandemic strain. The influenza virus may be a reassortant strain, and may have been obtained by reverse genetics techniques. The virus may be attenuated. The virus may be temperature-sensitive. The virus may be cold-adapted. About 15 μg of HA per strain is typical for use in vaccines, although lower doses (e.g. ≦10, ≦7.5, ≦5 μg HA per strain) can also be used.
A meningococcal protein. Genome sequences for meningococcal serogroups A [102] and B [103,104] have been reported, and suitable antigens can be selected from the encoded polypeptides [e.g. refs. 105-110]. Preferred compositions include one or more of the following five antigens [111]: (1) a ‘NadA’ protein, preferably in oligomeric form (e.g. in trimeric form); (2) a ‘741’ protein; (3) a ‘936’ protein; (4) a ‘953’ protein; and (5) a ‘287’ protein. Other antigens for inclusion include Hsf adhesin and/or a transferrin-binding protein such as TbpB [112] and/or NspA.
An outer membrane vesicle (OMV) preparation from meningococcus. The term “OMV” includes any proteoliposomic vesicle obtained by disrupting a bacterial outer membrane to form vesicles of the outer membrane that include protein components of the outer membrane. OMVs are prepared artificially from bacteria (e.g. by detergent treatment, or by non-detergent means). The term also encompasses blebs, microvesicles (MVs [113]) and ‘native OMVs’ (‘NOMVs’ [114]), which are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 115 & 116 describe Neisseria with high MV production. OMVs can be prepared in various ways. Methods for obtaining suitable preparations are disclosed in, for instance, the references cited herein. Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [117 & 118] being preferred for treating Neisseria) at a pH sufficiently high not to precipitate the detergent. The strain used for OMV preparation may have been modified e.g. to have a modified fur gene [119], with nspA expression up-regulated and concomitant porA and cps knockout [120], or as described in references 121 to 125. OMVs may be supplemented with additional proteins e.g. see references 126 & 127. The OMVs are preferably obtained from one of the following meningococcal serosubtypes: P1.7b,4; P1.7,16; P1.19,15.
A Streptococcus pneumoniae protein. Genome sequences for several strains of pneumococcus are available [128,129] and can be subjected to reverse vaccinology [130-133] to identify suitable polypeptide antigens [134,135]. For example, the composition may include one or more of the following antigens: PhtA, PhtD, PhtB, PhtE, SpsA, LytB, LytC, LytA, Sp125, Sp101, Sp128, Sp130 and Sp130, as defined in reference 136. The composition may include more than one (e.g. 2, 3, 4, 5, 6, 7, 8, 9 10, 11, 12, 13 or 14) of these antigens.
A Streptococcus agalactiae protein, such as those disclosed in references 137 and 138.
A Streptococcus pyogenes antigen e.g. as disclosed in references 138, 139 and 140.
A Moraxella catarrhalis antigen e.g. as disclosed in reference 141.
A Staphylococcus aureus antigen e.g. as disclosed in reference 142.
A measles virus antigen mumps virus antigen and/or rubella virus antigen. Antigens for protecting against measles, mumps and rubella viruses are typically live viruses, as found in known monovalent and trivalent (‘MMR’) vaccines. Measles virus vaccines are described in more detail in chapter 19 of reference 88. Mumps virus vaccines are described in more detail in chapter 20 of reference 88. Rubella virus vaccines are described in more detail in chapter 26 of reference 88. Typical measles virus strains include: Moraten; Connaught; Schwarz; Edmonston-Zagreb; CAM-70; AIK-C; TD97; Leningrad-16; Shanghai-191; etc. The Schwarz and Moraten strains are most common for use in USA and Europe. Typical mumps virus strains include: Jeryl Lynn; RIT 4385; Urabe; Hoshino; Rubini; Leningrad-3; Leningrad-Zagreb; Miyahara; Torii; NK M-46; S-12; etc. The Jeryl Lynn, RIT 4385, Urabe and Leningrad-Zagreb strains are the most common worldwide strains. Typical rubella virus strains include: RA27/3; Matsuba; TCRB 19; Takahashi; Matsuura; TP-336; etc. The RA27/3 strain is the most common strain used in the western world.
A varicella zoster virus antigen for protecting against chickenpox. These are typically live viruses, based on the Oka strain of the virus. VZV vaccines are described in more detail in chapter 28 of reference 88.

The invention is particularly suitable for use with conjugated saccharide antigens, with antibody titres exceeding those seen with aluminium salts. Suitable saccharide antigens include but are not limited to conjugated capsular saccharides from the following bacteria:
Haemophilus influenzae type B (‘Hib’). Hib conjugates are disclosed in more detail in chapter 14 of reference 88. The saccharide moiety of a Hib conjugate may comprise full-length polyribosylribitol phosphate (PRP) as prepared from Hib bacteria, or it may comprise fragments of full-length PRP. The amount of Hib conjugate, measured as saccharide, in compostions of the invention is typically between 10 and 30 μg/ml. Administration of the Hib conjugate preferably results in an anti-PRP antibody concentration of ≧0.15 μg/ml, and more preferably ≧1 μg/ml, and these are the standard acceptable response thresholds.
Neisseria meningitidis serogroup C (‘MenC’). Conjugate vaccines against MenC have been approved for human use, and include MENJUGATE™ [143], MENINGITEC™ and NEISVAC-C™. Serogroup C saccharides may be prepared from either OAc+ or OAc− strains.
Neisseria meningitidis serogroup A (‘MenA’). Preferably at least 50% (e.g. at least 60%, 70%, 80%, 90%, 95% or more) of the mannosamine residues are O-acetylated at the C-3 position.
Neisseria meningitidis serogroup W135 (‘MenW135’).
Neisseria meningitidis serogroup Y (‘MenY’).
Streptococcus pneumoniae, e.g. refs. 144 to 146. It is preferred to include saccharides from more than one serotype of S. pneumoniae: mixtures of polysaccharides from 23 different serotype are widely used, as are conjugate vaccines with polysaccharides from between 5 and 11 different serotypes [147]. For example, PrevNar™ [148] contains antigens from seven serotypes (4, 6B, 9V, 14, 18C, 19F, and 23F) with each saccharide individually conjugated to CRM197 by reductive amination, with 2 μg of each saccharide per 0.5 ml dose (4 μg of serotype 6B). Compositions of the invention preferably include at least serotypes 6B, 14, 19F and 23F. Further serotypes are preferably selected from: 1, 3, 4, 5, 7F, 9V and 18C. The amount of a pneumococcal conjugate, measured as saccharide, in compositions of the invention is typically between 2 and 20 μg/ml for each serotype.
Streptococcus pyogenes (‘GAS’) e.g. as described in reference 149.
Streptococcus agalactiae (‘GBS’) e.g. as described in references 150-154. Saccharides from GBS serotypes Ia, Ib and/or III will typically be included. GBS serotypes IV, V and VII may also be used.

For immunising against meningococcus, it is preferred to include saccharides from more than one serogroup. Mixtures of conjugates from serogroups A+C are known [155,156] and mixtures of conjugates from serogroups A+C+W135+Y have been reported [157-160] and were approved in 2005 as the MENACTRA™ product. The meningococcal saccharide(s) used in the invention can be from one or more of serogroups A, C, W135 and Y e.g. A+C, A+W135, A+Y, C+W135, C+Y, W135+Y, A+C+W135, A+C+Y, C+W135+Y, A+C+W135+Y. It is preferred to use at least the serogroup C saccharide. The saccharide moieties of the conjugates may comprise full-length saccharides as prepared from meningococci, and/or it fragments of full-length saccharides. The amount of a meningococcal conjugate, measured as saccharide, in compositions of the invention is typically between 5 and 25 μg/ml for each serogroup. Administration of a conjugate preferably results in an increase in serum bactericidal assay (SBA) titre for the relevant serogroup of at least 4-fold, and preferably at least 8-fold. SBA titres can be measured using baby rabbit complement or human complement [161].

The invention is particularly suitable for use with conjugated saccharides, with appropriate buffers being used to enhance adsorption. Even if these buffers do not enhance adsorption of a particular non-conjugated antigen, their use is advantageous because it allows the composition to be combined with the buffered conjugate compositions without changing the buffer system (i.e. where the two compositions use the same buffer).

When making multivalent combinations, antigens can be combined individually in series, or they can be pre-mixed and added together. Antigenic components can be combined in any suitable order.

Preferred compositions comprising multiple antigens may comprise: a mixture of diphtheria, tetanus and pertussis antigens; a mixture of diphtheria and tetanus antigens; a mixture of diphtheria, tetanus, pertussis and HBsAg antigens; a mixture of diphtheria, tetanus, pertussis and inactivated poliovirus antigens; a mixture of diphtheria, tetanus, pertussis, HBsAg and inactivated poliovirus antigens; a mixture of Hib and one or more meningococcal conjugates; etc. References 12 & 162 compared aluminum hydroxide and calcium phosphate as the adjuvant for bivalent diphtheria-tetanus vaccines (see also refs. 163 & 164).

### Pharmaceutical Compositions

In addition to the adjuvant and antigen components, compositions of the invention may include further components. These components may have various sources. For example, they may be present in one of the antigen or adjuvant components that is used during manufacture or may be added separately from the antigenic components.

Preferred compositions of the invention include one or more pharmaceutical carrier(s) and/or excipient(s).

To control tonicity, it is preferred to include a physiological salt, such as a sodium salt. Sodium chloride (NaCl) is preferred, which may be present at between 1 and 20 mg/ml.

Compositions will generally have an osmolality of between 200 mOsm/kg and 400 mOsm/kg, preferably between 240-360 mOsm/kg, and will more preferably fall within the range of 290-310 mOsm/kg.

Compositions of the invention may include one or more buffers. Typical buffers include: a phosphate buffer, such as a sodium phosphate buffer; a Tris buffer; a borate buffer; a succinate buffer; a histidine buffer; or a citrate buffer. Buffers will typically be included in the 2-20 mM range. The inclusion of a histidine buffer, for instance, has been found to enhance the level of antigen adsorption to calcium phosphate.

The pH of a composition of the invention will generally be between 5.5 and 7.5, or between 6.0 and 7.0. A process of the invention may therefore include a step of adjusting pH prior to packaging.

Compositions of the invention are preferably sterile.

Compositions of the invention are preferably non-pyrogenic e.g. containing <1 EU (endotoxin unit, a standard measure) per dose, and preferably <0.1 EU per dose.

Compositions of the invention are preferably gluten free.

Due to the adsorbed nature of antigens, the final vaccine product may be a suspension with a cloudy appearance. This appearance means that microbial contamination is not readily visible, and so the vaccine preferably contains a preservative. This is particularly important when the vaccine is packaged in multidose containers. Although a typical preservative used in vaccines is thimerosal, with the invention it is preferred not to use mercurial preservatives. However, the presence of trace amounts may be unavoidable if a bulk antigen was treated with such a preservative before being used to prepare the composition of the invention. For safety, however, it is preferred that the final composition contains <10 μg/ml mercury, more preferably <1 μgml, and most preferably <100 ng/ml. Instead of mercurial preservatives, it is preferred to use 2-phenoxyethanol.

During manufacture, dilution of components to give desired final concentrations will usually be performed with WFI (water for injection).

Compositions of the invention are preferably administered to patients in 0.5 ml doses. References to 0.5 ml doses will be understood to include normal variance e.g. 0.5 ml±0.05 ml.

The invention can provide bulk material which is suitable for packaging into individual doses, which can then be distributed for administration to patients. Concentrations mentioned above are typically concentrations in final packaged dose, and so concentrations in bulk vaccine may be higher (e.g. to be reduced to final concentrations by dilution).

Compositions of the invention will generally be in aqueous form.

### Packaging Compositions of the Invention

After combining antigen(s) and adjuvant(s), a process of the invention may comprise a step of extracting and packaging a sample (e.g. a 0.5 ml sample) of the mixture into a container.

A process of the invention may comprise the further step of packaging the vaccine into containers for use. Suitable containers include vials and disposable syringes (preferably sterile ones).

Where a composition of the invention is packaged into vials, these are preferably made of a glass or plastic material. The vial is preferably sterilized before the composition is added to it. To avoid problems with latex-sensitive patients, vials are preferably sealed with a latex-free stopper. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass. Where the composition is packaged into a syringe, the syringe will not normally have a needle attached to it, although a separate needle may be supplied with the syringe for assembly and use. Safety needles are preferred. Disposable syringes contain a single dose of vaccine.

Where a glass container (e.g. a syringe or a vial) is used, then it is preferred to use a container made from a borosilicate glass rather than from a soda lime glass.

After a composition is packaged into a container, the container can then be enclosed within a box for distribution e.g. inside a cardboard box, and the box will be labeled with details of the vaccine.

The vaccine may be packaged together (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

The packaged vaccine is preferably stored at between 2° C. and 8° C. It should not be frozen.

Vaccines can be provided in full-liquid form (i.e. where all antigenic components are in aqueous solution or suspension) during manufacture, or they can be prepared in a form where some components are in liquid form and others are in a lyophilized form. Thus a final vaccine can be prepared extemporaneously at the time of use by mixing together two components: (a) a first component comprising aqueous antigens; and (b) a second component comprising lyophilized antigens. The two components are preferably in separate containers (e.g. vials and/or syringes), and the invention provides a kit comprising components (a) and (b). Lyophilized components may include stabilizers such as lactose, sucrose or mannitol, as well as mixtures thereof e.g. lactose/sucrose mixtures, sucrose/mannitol mixtures, etc.

### Methods of Treatment, and Administration of the Vaccine

Compositions of the invention are suitable for administration to human patients, and the invention provides a method of raising an immune response in a patient, comprising the step of administering a composition of the invention to the patient.

The invention also provides a composition of the invention for use in medicine.

The invention also provides the use of (i) an antigen and (ii) a calcium phosphate antigen, in the manufacture of a medicament for administering to a patient.

Immunogenic compositions of the invention are preferably vaccines, for use in the prevention and/or treatment of infections caused by the pathogens whose antigens are included in the compositions.

Compositions of the invention can be administered by intramuscular injection e.g. into the arm or leg

In order to have full efficacy, a typical immunization schedule for a child may involve administering more than one dose. For example, doses may be at: 0 & 6 months (time 0 being the first dose); at 0, 1, 2 & 6 months; at day 0, day 21 and then a third dose between 6 & 12 months; or at 0, 1, 2, 6 & 12 months.

Settling of components may occur during storage. The composition should therefore be shaken prior to administration to a patient. The shaken composition will be a turbid white suspension.

### Carrier Proteins for Conjugates

Conjugated saccharide antigens include a carrier protein, to which the saccharide is covalently attached, either directly or via a linker. General information on conjugation techniques can be found in reference 165.

Various proteins are known for use as carriers, and preferred carrier proteins are bacterial toxins or toxoids, such as diphtheria toxoid or tetanus toxoid. Other suitable carrier proteins include, but are not limited to, the CRM197 mutant of diphtheria toxin [166-168], the N. meningitidis outer membrane protein [169], synthetic peptides [170, 171], heat shock proteins [172,173], pertussis proteins [174,175], cytokines [176], lymphokines [176], hormones [176], growth factors [176], artificial proteins comprising multiple human CD4⁺ T cell epitopes from various pathogen-derived antigens [177] such as N19 [178], protein D from H. influenzae [179,180], pneumococcal surface protein PspA [181], pneumolysin [182], iron-uptake proteins [183], toxin A or B from C. difficile [184], S. agalactiae proteins [185], etc.

Attachment of a saccharide to a carrier is preferably via a —NH₂ group e.g. in the side chain of a lysine residue in a carrier protein, or of an arginine residue. Attachment to —SH groups (e.g. in the side chain of a cysteine) is also possible.

Conjugates with a saccharide:protein ratio (w/w) of between 1:5 (i.e. excess protein) and 5:1 (i.e. excess saccharide) are preferred.

Compositions may include a small amount of free carrier. Ignoring any carrier included as a separate antigen, unconjugated carrier is preferably no more than 5% of the total amount of the carrier protein in the composition as a whole, and more preferably present at less than 2% by weight.

It is possible to include more than one type of carrier protein in a composition e.g. to reduce the risk of carrier suppression.

The MENJUGATE™ and MENINGITEC™ products use a CRM197 carrier protein, and this carrier can also be used according to the invention. The NEISVAC-C™ product uses a tetanus toxoid carrier protein, and this carrier can also be used according to the invention, as can diphtheria toxoid.

Hib conjugates preferably use a CRM197 or tetanus toxoid carrier protein.

### General

The term “comprising” encompasses “including” as well as “consisting” e.g. a composition “comprising” X may consist exclusively of X or may include something additional e.g. X+Y. The word substantially does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE).

### MODES FOR CARRYING OUT THE INVENTION

Superfos calcium phosphate adjuvant was obtained from Brenntag Biosector in Denmark, and was used for adjuvanting the following antigens: (1) diphtheria toxoid; (2) tetanus toxoid; (3) protein ‘287’ from serogroup B meningococcus; (4) HBsAg; (5) serogroup C meningococcus capsular saccharide, conjugated to CRM197; (6) a mixture of meningococcus capsular saccharides, conjugated to CRM197, from serogroups C, W135 and Y; (7) a hybrid ‘741’ protein from serogroup B meningococcus; (8) a mixture of a hybrid ‘741’ protein, NadA and a 287/953 hybrid from serogroup B meningococcus.

An aluminium phosphate adjuvant was also obtained from Brenntag Biosector for comparison.

Some compositions included a sodium phosphate or histidine buffer. Sodium chloride was included in all of the compositions at 9 mg/ml.

The final pH of the compositions was measured, as was the % of antigen that was adsorbed to the metal salt adjuvant. Results of the analysis are shown in Table I. Adsorption levels were very high, reaching up to 100% (i.e. no antigen detectable in supernatant after centrifugation).

Osmolarity of various compositions for antigens (1), (2) and (3) was measured and fell into the range of 283 to 297 mOsm/kg.

Table I shows % adsorption at time zero. Adsorption of antigens (1), (2) and (3) was also measured after 2 weeks of storage at either 2-8° C. or 36-28° C. Results were as follows:

| | | | % adsorption | | |
|---|---|---|---|---|---|
| | | | | 2 wk, | 2 wk, |
| Antigen | [Antigen] | Adjuvant | Time zero | 2-8° C. | 36-38° C. |
| (1) | 5 | AlH | ~100 | ~100 | ~100 |
| (1) | 50 | AlH | >97.5 | >97.5 | ~99 |
| (1) | 5 | CaP | ~100 | ~100 | ~100 |
| (1) | 50 | CaP | >95 | >95 | ~99 |
| (2) | 5 | AlH | ~100 | ~100 | ~100 |
| (2) | 50 | AlH | >97.5 | >97.5 | ~99 |
| (2) | 5 | CaP | ~100 | ~100 | ~100 |
| (2) | 50 | CaP | >95 | >95 | ~99 |
| (3) | 20 | AlH | ~100 | ~100 | ~100 |
| (3) | 200 | AlH | >97.5 | >97.5 | ~99 |
| (3) | 20 | CaP | ~100 | ~100 | ~100 |
| (3) | 200 | CaP | >95 | >95 | ~99 |

Thus adsorption remained high, and was even seen to increase during storage at 36-38° C.

The calcium phosphate adjuvant was characterised and found to have a mean particle size of 6-7.5 μm and a zeta potential of −12±3 mV.

In immunological studies, the CaP-adjuvanted compositions generally elicited lower immune responses than the AlH-adjuvanted compositions. For the MenC conjugate, however, the ELISA titre was higher with the CaP adjuvant, although the SBA titre was lower.

It will be understood that the invention has been described by way of example only and modifications may be made whilst remaining within the scope and spirit of the invention.

TABLE I

| | | | | Buffer | Final | |
|---|---|---|---|---|---|---|
| Antigen | [Antigen] | Adjuvant | Buffer | pH | pH | % |
| (1) | 50 | CaP | (a) | 6.80 | 6.67 | >97.5 |
| (1) | 50 | CaP | (b) | 6.50 | 6.56 | >97.5 |
| (1) | 50 | CaP | — | — | 6.51 | >97.5 |
| (1) | 50 | AlH | (a) | 6.80 | 6.80 | >97.5 |
| (1) | 50 | AlH | (b) | 6.50 | 6.48 | >97.5 |
| (1) | 50 | AlH | — | — | 6.12 | >97.5 |
| (1) | 5 | CaP | (b) | 6.50 | 6.54 | >97.5 |
| (1) | 5 | CaP | — | — | 6.49 | >97.5 |
| (1) | 5 | AlH | — | — | 5.96 | >97.5 |
| (2) | 50 | CaP | (a) | 6.80 | 6.67 | >97.5 |
| (2) | 50 | CaP | (b) | 6.50 | 6.55 | >97.5 |
| (2) | 50 | CaP | — | — | 6.52 | >97.5 |
| (2) | 50 | AlH | (a) | 6.80 | 6.84 | >97.5 |
| (2) | 50 | AlH | (b) | 6.50 | 6.45 | >97.5 |
| (2) | 50 | AlH | — | — | 5.88 | >97.5 |
| (2) | 5 | CaP | (b) | 6.50 | 6.54 | ~100 |
| (2) | 5 | CaP | — | — | 6.48 | ~100 |
| (2) | 5 | AlH | — | — | 5.81 | ~100 |
| (3) | 200 | CaP | (b) | 6.50 | 6.61 | ~98 |
| (3) | 200 | AlH | (b) | 6.50 | 6.52 | >90 |
| (3) | 20 | CaP | (b) | 6.53 | 6.52 | ~100 |
| (3) | 20 | AlH | (b) | 6.46 | 6.52 | ~100 |
| (4) | 50 | CaP | (b) | 6.50 | 6.66 | ~100 |
| (4) | 50 | AlH | (c) | 6.60 | 6.73 | ~100 |
| (4) | 5 | CaP | (b) | 6.50 | 6.55 | ~100 |
| (4) | 5 | AlH | (c) | 6.60 | 6.64 | ~100 |
| (5) | 100 | AlH | (b) | 7.20 | 6.95 | 99 |
| (5) | 100 | CaP | (c) | 7.20 | 7.00 | 54 |
| (5) | 100 | CaP | (b) | 7.20 | 6.82 | 83 |
| (5) | 10 | AlH | (b) | 7.20 | 6.84 | 100 |
| (5) | 10 | CaP | (b) | 7.20 | 6.83 | 100 |
| (6) | 20 | CaP | (b) | 7.20 | 6.83 | 99.6 |
| (7) | 100 | CaP | (d) | 7.20 | 6.05 | ~100 |
| (8) | 50 | CaP | (d) | 6.00 | 6.73 | 94-100⁽*⁾ |

Antigen concentrations are in Lf/ml (Dt and Tt) or μg/ml (all other antigens). Values are per antigen.
Adjuvant was (i) CaP, calcium phosphate at 1 mg of Ca⁺⁺ per ml, or (ii) AlH, aluminium hydroxide at 2 mg salt per ml (~0.7 mg Al⁺⁺⁺ per ml).
Buffers are:
(a) 5 mM Na phosphate;
(b) 5 mM histidine;
(c) 10 mM Na phosphate; (d) 10 mM histidine
% is percentage of antigen in pellet after centrifugation.
⁽*⁾hybrid ‘741’ was 94%, NadA was ~100%, 287/953 hybrid was 98%.

### REFERENCES

### The Contents of which are Hereby Incorporated by Reference

[1] U.S. Pat. No. 2,967,802.
[2] Martin et al. (1969) Presse Med 77(10):341-4.
[3] Vaccine Design . . . (1995) eds. Powell & Newman. ISBN: 030644867X. Plenum.
[4] Jiang et al. (2004) Vaccine 23:693-8.
[5] U.S. Pat. No. 5,676,976.
[6] WO00/46147.
[7] U.S. Pat. No. 6,355,271.
[8] WO03/051394.
[9] U.S. Pat. No. 5,443,832.
[10] U.S. Pat. No. 5,851,670.
[11] U.S. Pat. No. 4,016,252.
[12] Aggerbeck & Heron (1995) Vaccine 13:1360-5.
[13] WO00/15194.
[14] WO00/37045.
[15] Kandimalla et al. (2003) Nucleic Acids Research 31:2393-2400.
[16] WO02/26757.
[17] WO99/62923.
[18] Krieg (2003) Nature Medicine 9:831-835.
[19] McCluskie et al. (2002) FEMS Immunology and Medical Microbiology 32:179-185.
[20] WO98/40100.
[21] U.S. Pat. No. 6,207,646.
[22] U.S. Pat. No. 6,239,116.
[23] U.S. Pat. No. 6,429,199.
[24] Kandimalla et al. (2003) Biochemical Society Transactions 31 (part 3):654-658.
[25] Blackwell et al. (2003) J Immunol 170:4061-4068.
[26] Krieg (2002) Trends Immunol 23:64-65.
[27] WO01/95935.
[28] Kandimalla et al. (2003) BBRC 306:948-953.
[29] Bhagat et al. (2003) BBRC 300:853-861.
[30] WO03/035836.
[31] Johnson et al. (1999) Bioorg Med Chem Lett 9:2273-2278.
[32] Evans et al. (2003) Expert Rev Vaccines 2:219-229.
[33] Baldridge et al. (2002) J Endotoxin Res 8:453-8.
[34] Persing et al. (2002) Trends Immunol 10:S32-S37.
[35] UK patent application 2,220,211.
[36] Myers et al. (1990) pages 145-156 of Cellular and molecular aspects of endotoxin reactions.
[37] Ulrich (2000) Chapter 16 (pages 273-282) of Vaccine Adjuvants: Preparation Methods and Research Protocols (Volume 42 of Methods in Molecular Medicine series). ISBN: 1-59259-083-7. Ed. O'Hagan.
[38] Johnson et al. (1999) J Med Chem 42:4640-9.
[39] Baldrick et al. (2002) Regulatory Toxicol Pharmacol 35:398-413.
[40] Meraldi et al. (2003) Vaccine 21:2485-2491.
[41] Pajak et al. (2003) Vaccine 21:836-842.
[42] WO90/14837.
[43] U.S. Pat. No. 5,057,540.
[44] WO96/33739.
[45] EP-A-0,109,942.
[46] WO96/11711.
[47] WO00/07621.
[48] Barr et al. (1998) Advanced Drug Delivery Reviews 32:247-271.
[49] Sjolanderet et al. (1998) Advanced Drug Delivery Reviews 32:321-338.
[50] Niikura et al. (2002) Virology 293:273-280.
[51] Lenz et al. (2001) J Immunol 166:5346-5355.
[52] Pinto et al. (2003) J Infect Dis 188:327-338.
[53] Gerber et al. (2001) Virol 75:4752-4760.
[54] WO03/024480
[55] WO03/024481
[56] Gluck et al. (2002) Vaccine 20:B10-B16.
[57] WO95/17211.
[58] WO98/42375.
[59] Beignon et al. (2002) Infect Immun 70:3012-3019.
[60] Pizza et al. (2001) Vaccine 19:2534-2541.
[61] Pizza et al. (2000) Int J Med Microbiol 290:455-461.
[62] Scharton-Kersten et al. (2000) Infect Immun 68:5306-5313.
[63] Ryan et al. (1999) Infect Immun 67:6270-6280.
[64] Partidos et al. (1999) Immunol Lett 67:209-216.
[65] Peppoloni et al. (2003) Expert Rev Vaccines 2:285-293.
[66] Pine et al. (2002) J Control Release 85:263-270.
[67] Domenighini et al. (1995) Mol Microbiol 15:1165-1167.
[68] WO99/40936.
[69] WO99/44636.
[70] Singh et al] (2001) J Cont Release 70:267-276.
[71] WO99/27960.
[72] U.S. Pat. No. 6,090,406
[73] U.S. Pat. No. 5,916,588
[74] EP-A-0,626,169.
[75] WO99/52549.
[76] WO01/21207.
[77] WO01/21152.
[78] Andrianov et al. (1998) Biomaterials 19:109-115.
[79] Payne et al. (1998) Adv Drug Delivery Review 31:185-196.
[80] U.S. Pat. No. 4,680,338.
[81] U.S. Pat. No. 4,988,815.
[82] WO92/15582.
[83] Stanley (2002) Clin Exp Dermatol 27:571-577.
[84] Wu et al. (2004) Antiviral Res. 64(2):79-83.
[85] Vasilakos et al. (2000) Cell Immunol. 204(1):64-74.
[86] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[87] Jones (2003) Curr Opin Investig Drugs 4:214-218.
[88] Vaccines. (eds. Plotkin & Orenstein). 4th edition, 2004, ISBN: 0-7216-9688-0.
[89] Sesardic et al. (2001) Biologicals 29:107-22.
[90] NIBSC code: 98/560.
[91] Module 1 of WHO's The immunological basis for immunization series (Galazka).
[92] NIBSC code: 69/017.
[93] NIBSC code: DIFT.
[94] Sesardic et al (2002) Biologicals 30:49-68.
[95] NIBSC code: 98/552.
[96] NIBSC code: TEFT.
[97] NIBSC code: 66/303.
[98] Rappuoli et al. (1991) TIBTECH 9:232-238.
[99] Module 6 of WHO's The immunological basis for immunization series (Robertson)
[100] Audsley & Tannock (2004) Expert Opin Biol Ther. 4:709-17.
[101] U.S. Pat. No. 5,948,410.
[102] Parkhill et al. (2000) Nature 404:502-506.
[103] Tettelin et al. (2000) Science 287:1809-1815.
[104] WO00/66791.
[105] Pizza et al. (2000) Science 287:1816-1820.
[106] WO99/24578.
[107] WO99/36544.
[108] WO99/57280.
[109] WO00/22430.
[110] WO00/66741.
[111] WO2004/032958.
[112] WO2004/014419.
[113] WO02/09643.
[114] Katial et al. (2002) Infect. Immun. 70:702-707.
[115] U.S. Pat. No. 6,180,111.
[116] WO01/34642.
[117] European patent 0,011,243.
[118] Fredriksen et al. (1991) NIPH Ann. 14(2):67-80.
[119] WO98/56901.
[120] WO02/09746.
[121] WO01/09350.
[122] WO02/062378.
[123] WO2004/014417.
[124] WO2004/019977.
[125] WO2004/048404.
[126] WO00/25811.
[127] WO01/52885.
[128] Tettelin et al. (2001) Science 293:498-506.
[129] Hoskins et al (2001) J Bacteriol 183:5709-5717.
[130] Rappuoli (2000) Curr Opin Microbiol 3:445-450
[131] Rappuoli (2001) Vaccine 19:2688-2691.
[132] Masignani et al. (2002) Expert Opin Biol Ther 2:895-905.
[133] Mora et al. (2003) Drug Discov Today 8:459-464.
[134] Wizemann et al. (2001) Infect Immun 69:1593-1598.
[135] Rigden et al. (2003) Crit Rev Biochem Mol Biol 38:143-168.
[136] WO02/22167.
[137] Schuchat (1999) Lancet 353(9146):51-6.
[138] WO02/34771.
[139] Dale (1999) Infect Dis Clin North Am 13:227-43, viii.
[140] Ferretti et al. (2001) PNAS USA 98: 4658-4663.
[141] McMichael (2000) Vaccine 19 Suppl 1:S101-107.
[142] Kuroda et al. (2001) Lancet 357(9264):1225-1240; see also pages 1218-1219.
[143] Jones (2001) Curr Opin Investig Drugs 2:47-49.
[144] Watson (2000) Pediatr Infect Dis J 19:331-332.
[145] Rubin (2000) Pediatr Clin North Am 47:269-285, v.
[146] Jedrzejas (2001) Microbiol Mol Biol Rev 65:187-207.
[147] Zielen et al. (2000) Infect. Immun. 68:1435-1440.
[148] Darkes & Plosker (2002) Paediatr Drugs 4:609-630.
[149] Sabharwal et al. (2006) J Infect Dis 193:129-35.
[150] Baker & Edwards (2003) Arch Dis Child 88:375-8.
[151] Paoletti & Kasper (2002) J Infect Dis 186:126-6.
[152] Palazzi et al. (2004) J Infect Dis 190:558-64.
[153] Heath & Feldman (2005) Expert Rev Vaccines 4:207-18.
[154] Paoletti & Kasper (2003) Expert Opin Biol Ther 3:975-84.
[155] Costantino et al. (1992) Vaccine 10:691-8.
[156] Lieberman et al. (1996) JAMA 275:1499-503.
[157] WO02/058737.
[158] WO03/007985.
[159] Rennels et al. (2002) Pediatr Infect Dis J 21:978-979.
[160] Campbell et al. (2002) J infect Dis 186:1848-1851.
[161] WHO. Tech. Rep. Ser. 594:51, 1976.
[162] Aggerbeck & Heron (1995) Vaccine 13:1366-74.
[163] Aggerbeck & Heron (1995) Dev Biol Stand 86:297-301.
[164] Aggerbeck et al. (1996) Vaccine 14:1265-72.
[165] Conjugate Vaccines (eds. Cruse et al.) ISBN 3805549326, particularly vol. 10:48-114.
[166] Anonymous (January 2002) Research Disclosure, 453077.
[167] Anderson (1983) Infect Immun 39(1):233-238.
[168] Anderson et al. (1985) J Clin Invest 76(1):52-59.
[169] EP-A-0,372,501.
[170] EP-A-0,378,881.
[171] EP-A-0,427,347.
[172] WO93/17712
[173] WO94/03208.
[174] WO98/58668.
[175] EP-A-0,471,177.
[176] WO91/01146
[177] Falugi et al (2001) Eur J Immunol 31:3816-24.
[178] Baraldo et al. (2004) Infect Immun 72:4884-87.
[179] EP-A-0,594,610.
[180] WO00/56360.
[181] WO02/091998.
[182] Kuo et al. (1995) Infect Immun 63:2706-13.
[183] WO01/72337
[184] WO00/61761.
[185] WO2004/041157.

1. An immunogenic composition comprising: (i) an antigen; and (ii) a calcium phosphate salt, wherein at least 80% of the antigen is adsorbed to the calcium phosphate.

2. The composition of claim 1, wherein the calcium phosphate is amorphous.

3. The composition of any preceding claim, wherein the calcium phosphate is in particulate form.

4. The composition of any preceding claim, wherein the calcium phosphate has a calcium to phosphorus molar ratio between 1.35 and 1.83.

5. The composition of any preceding claim, wherein the concentration of calcium phosphate, measured as Ca⁺⁺, is between 0.1 mg/ml and 10 mg/ml.

6. The composition of any preceding claim, wherein at least 90% of the antigen is adsorbed.

7. The composition of claim 6, wherein at least 95% of the antigen is adsorbed.

8. The composition of claim 7, wherein at least 99% of the antigen is adsorbed.

9. The composition of any preceding claim, including one or more further adjuvants and/or immunostimulatory agents.

10. The composition of claim 9, including an immunostimulatory oligonucleotide.

11. The composition of any preceding claim, which is substantially free from aluminium salts.

12. The composition of any preceding claim, wherein the antigen is a bacterial or viral antigen.

13. The composition of claim 12, wherein the antigen is a conjugated bacterial capsular saccharide.

14. The composition of claim 13, wherein the capsular saccharide is from H. influenzae type B.

15. The composition of claim 13, wherein the capsular saccharide is from N. meningitidis.

16. The composition of claim 13, wherein the capsular saccharide is from S. pneumoniae.

17. The composition of claim 12, wherein the antigen is an influenza virus antigen.

18. The composition of claim 17, wherein the influenza virus is a pandemic strain.

19. The composition of any preceding claim, wherein the composition includes NaCl.

20. The composition of any preceding claim, wherein the composition has an osmolality between 200 mOsm/kg and 400 mOsm/kg.

21. The composition of any preceding claim, wherein the composition includes a buffer.

22. The composition of claim 21, wherein the composition includes a histidine buffer.

23. The composition of any preceding claim, wherein the composition has a pH between 5.5 and 7.5.

24. The composition of any preceding claim, wherein the composition is free from mercury.

25. An adjuvant composition comprising: (i) a calcium phosphate salt; and (ii) an adjuvant selected from the group consisting of: 3D-MPL, immunostimulatory oligonucleotides, and imidazoquinolones; wherein at least 50% of the adjuvant is adsorbed to the calcium phosphate.
